(19) United States
(12) Patent Application Publication
(10) Pub. No.: US 2022/0313343 A1
(43) Pub. Date: Oct. 6, 2022

(54) OPERATION OF AN ELECTROSURGICAL GENERATOR

(71) Applicant: OLYMPUS WINTER & IBE GMBH, Hamburg (DE)

(72) Inventors: Jens KRÜGER, Zeuthen (DE); Fabian JANICH, Potsdam (DE)

(73) Assignee: OLYMPUS WINTER & IBE GMBH, Hamburg (DE)

(21) Appl. No.: 17/705,601

(22) Filed: Mar. 28, 2022

(30) Foreign Application Priority Data
Mar. 31, 2021 (DE) 10 2021 108 190.4

(51) Int. Cl. A61B 18/12; A61B 34/00; G06F 8/65; G16H 80/00

(52) U.S. Cl. CPC A61B 18/1206; A61B 34/25; G06F 8/65; G16H 80/00; A61B 2034/256; A61B 2018/00892

(57) ABSTRACT

A method having: sending, to a spatially separately arranged server device, a configuration request having an item of identification information of the electrosurgical generator; receiving an item of operating information indicative of an operating mode, wherein the operating mode is capable of partially controlling the energy supply of the electrosurgical instrument by the electrosurgical generator; and storing the operating information in a data memory of the electrosurgical generator. Furthermore, a method for operating a server device, the method having: receiving a configuration request having an item of identification information of an electrosurgical generator; determining, at least partially in dependence on the identification information, an item of operating information indicative of one operating mode of a plurality of predefined operating modes, wherein operating modes are each capable of partially controlling an energy supply of an electrosurgical instrument by the electrosurgical generator; and sending, to the electrosurgical generator, the operating information.

The present invention relates to a method for operating an electrosurgical generator and a method for operating a server device, a corresponding computer program, and an electrosurgical generator, a server device, and an electrosurgical system.

In electrosurgery or high-frequency surgery, high-frequency alternating current is introduced into tissue of the human body by means of an electrosurgical instrument, such as an electroscalpel, in particular to cut or sever the tissue by way of the heating thus caused and to remove tissue in terms of a thermal resection. One advantage here is that hemostasis can also take place at the same time with the incision by clo-sure of the affected vessels and electrosurgical instruments come into consideration for further applications, for example, for coagulation.

Electrosurgical generators are used for the energy supply of electrosurgical instruments, so that they are medical devices in this case. Generators of this type are designed to emit a high-frequency AC voltage to an electrosurgical instrument. For this purpose, quite significant powers are required, and at frequencies of 100 kHz or higher, up to 4000 kHz, typically around 400 kHz. At such frequencies, the body tissue behaves like an ohmic resistor. The specific resistance is strongly dependent on the type of tissue, however, so that the specific resistances of muscles, fat, or bones differ strongly from one another, by up to a factor of 1000. This has the result that the load impedance of the electrical instrument can change strongly and quickly during operation in dependence on the tissue to be cut. This places special, characteristic demands on the electrosurgical generator and above all on its inverter. A rapid voltage regulation is thus typically necessary, in an environment having high voltages in the range of several kilovolts and high frequencies in the abovementioned range.

For the operation of these generators, providing different operating modes, for example, various profile forms of power, voltage, current, or frequency to be provided by the generator, is known, which can be used by the user for the energy supply of an electrosurgical instrument. All operating modes available at the point in time of startup are typically installed on the electrosurgical generator on a corresponding memory. Modifications of the electrosurgical generator and its operating modes by the user are not provided or are only provided with regard to individual operating parameters, not least due to the complex and extensive requirements for the authorization of medical devices. Exchanging operating modes or providing additional operating modes, for example newly developed profile forms, by the user is therefore not known in the prior art. Typically, the preinstalled operating modes can be changed—if at all—only by the producer, so that refine-ments and technical improvements generally cannot be made available to the user.

The operating modes installed on the generator are typically designed and authorized for use with specific electrosurgical instruments. The use of instruments, for example newly developed instruments, for the energy supply of which the existing operating modes of the generator are not provided, is therefore fundamentally not possible. This significantly restricts the user in the selection of electrosurgical instruments.

All of this significantly limits the effective service life of the electrosurgical generator and represents a significant disadvantage for the user-friendliness in the configuration and maintenance of the operational safety of an electrosurgical generator.

On the part of the producer, due to the complex and time-intensive development process in the development of: the de-sired operating modes, a long lead time is required until a generator having the provided operating modes can be put on the market. Short-term adjustments, for example, due to newly created specifications, can also only be performed with great effort and on location at the generator.

One object of the present invention is to provide, against the background of the abovementioned problems, improved methods for operating an electrosurgical generator and a server device.

The achievement of the object according to the invention lies in the features of the independent claims. Advantageous re-finements are the subject matter of the dependent claims.

Accordingly, the invention relates to a method for operating an electrosurgical generator for the energy supply of an electrosurgical instrument, the method comprising: sending, to a spatially separately % arranged server device, a configuration request comprising an item of identification information indicative of an identity of the electrosurgical generator; receiving, in particular from the server device, an item of operating information indicative of an operating mode, wherein the operating mode is capable of at least partially controlling the energy supply of the electrosurgical instrument by the electrosurgical generator; and storing the operating information in a data memory of the electrosurgical generator.

The invention furthermore relates to a method for operating a server device, the method comprising: receiving a configuration request, in articular sent by an electrosurgical generator, comprising an item of identification information indicative of an identity of the electrosurgical generator; determining, at least partially depending an the identification information, an item of operating information indicative of an operating mode of a plurality of predefined operating modes, wherein the operating modes are each capable of at least partially controlling an energy supply of an electrosurgical instrument by the electrosurgical generator; and sending the operating information to the electrosurgical generator.

The invention is also directed to an electrosurgical generator for the energy supply of an electrosurgical instrument having a processor, a communication interface, a data memory for storing one or more items of operating information, and a com-puter-readable data carrier, wherein instructions are stored in the data carrier which, upon execution by the processor, cause the method according to the invention for operating an electrosurgical generator to be carried out.

In addition, the invention is directed to a server device having a processor, a communication interface, a database for storing a plurality of items of operating information, and a computer-readable data carrier, wherein instructions are stored in the data carrier, which, upon execution by the processor, cause the method according no the invention for operating a server device to be carried out.

The invention is based on the finding that the effective service life of the electrosurgical generator and the long-term operational safety and the user-friendliness of the configuration and the operation of an electrosurgical generator can be increased in that an item of operating information is requested and provided, and operation can take place in dependence on this operating information. For this purpose, the operating information characterizes at least one operating mode custom-ized for the respective generator, adapt ed. thereto, and, for example, also authorized for it.

The user can thus if needed make use of further operating modes available for his electrosurgical generator and operate the generator, for example, using newly developed profile forms, which were not already present beforehand on his generator. A user, who has stored one or more individual and, for example, personalized operating modes on a server device, can also retrieve them independently of the respective generator to be used and operate the respective generator in accordance with the personalized operating modes. In this way, a physi-cian can make use of his preferred operating modes independently of the generator when changing between operating rooms or hospitals. A user can also in this way access operating modes of other users, who have made them available or dis-tributed them. The respective user can carry this out solely using the electrosurgical generator, for which purpose corresponding program software or an app can be installed on the generator, for example. For operation, the electrosurgical generator can then have, for example, a corresponding suitable user interface, such as a touch-sensitive display unit. In addition, the use of a token is conceivable, by which, inter aria, a further safety feature can be implemented for the operation of the electrosurgical generator.

Furthermore, operating modes for the energy supply of new electrosurgical instruments can also be provided after installation of the generator, for example in the case of an authorization of an instrument for use with the generator only after its installation. Additional operating modes, such as new profile forms, can thus be mace available promptly, for example, after completed authorization, for a generator already located in the field and can be sent later, for example, on request from a central server managed by the producer, without a complex producer service on location or a reauthorization of the hardware of the generator being required. Moreover, the possi-bility suggests itself of providing an electrosurgical generator having only a small number of permanently installed operating modes early, for which then newly developed operating modes can be received as needed by the user and operation in consideration of these operating modes is possible. Adapting the operation of an electrosurgical generator quickly to changing country-specific requirements is also made possible.

First, several concepts will be explained hereinafter:

An electrosurgical generator is to be understood as a device which is configured to supply an electrosurgical instrument with energy. An electrosurgical generator typically comprises a power supply unit, which feeds a DC voltage circuit in operation, and an inverter, which is fed by the DC voltage circuit and generates a high-frequency AC voltage, which is applied at outputs to the connection of an electrosurgical instrument. Typical electrosurgical instruments are electroknives and electroscalpels.

A server device can be both a single server and also multiple servers operatively connected to one another, for example ci server cloud.

The electrosurgical generator and the server device have at least one wireless and/or wired communication interface. By means of this communication interface, sending and/or receiving can take place, for example, via a wireless and/or wired communication connection. A communication connection is to be understood as a channel according to a communication technolo-gy, via which data can be sent and received, which comprise a representation of the operating information or the configuration request. The communication connection can be an at least partially secured communication connection. Exemplary communication connections are radio channels (known to a person skilled in the art, for example, under the acronyms RFID, NFC, Bluetooth, or WLAN), mobile data connections (known to a person skilled in the art, for example, under the acronyms GSM, UMTS, or LTE), or wired communication connections, which are known to a person skilled in the art, for example, as a local area network or as a bus system (for example, CAN bus or USE). The communication connection can comprise one or more nodes, for example routers, along which it at least partially ex-tends.

The data memory, the database, and the data carrier of the electrosurgical generator and the server device can be porta-ble or also integrated in the respective device. The database of the server device can be configured to store all available items of operating information. The data memory of the electrosurgical generator can store one or more items of operating information, which are a subset of the overall available items of operating information. Correspondingly, the one or more items of operating information stored in the data memory of the generator can only be indicative of a subset of all available operating modes.

A configuration request is to be understood as a request which a server device can receive and in dependence on which the server device can determine an item of operating information. This request comprises for this purpose at least one item of identification information. The identification information is indicative of the identity of an electrosurgical generator, for example for an electrosurgical generator of one design, and in particular for that generator which has sent the corresponding configuration request. For example, the identification information is indicative of an individual identity of an electrosurgical generator. The identification information can also be indicative of the current usage location of the electrosurgical generator, for the ascertainment of which the gen-erator comprises, for example, a suitable position sensor. Ad-ditionally, the identification information can be indicative of the identity of an electrosurgical instrument to be sup-plied with energy using the electrosurgical generator. For ex-ample, the identification information can represent a unique identifier of a generator and preferably also a unique or de-sign-specific identifier of an electrosurgical instrument.

An item of operating information is indicative of one operating mode of a plurality of predefined operating modes of the electrosurgical generator. This operating mode is represented by the corresponding operating information and may thus be assigned to a specific item of operating information. For exam-ple, for each of the plurality of operating modes, a represen-tation of the respective operating mode is stored in the data-base of the server device. Such representations can also each be stored in the data memory of the electrosurgical generator.

The operating mode can specify one or more operating parame-ters of the electrosurgical generator, for example powers, voltages, currents or frequencies to be provided. An operating parameter can be characterized, for example, by a measured variable.

The determination of an item of operating information is to be understood, for example, to mean that the operating infor-mation is selected at least partially in dependence on the identification information. For example, the determination can take place in consideration of the identity of the electrosur-gical generator and the identity of an electrosurgical instru-ment to be supplied with energy. For example, determination and/or selection rules are specified, which specify which operating mode is to be determined (for example selected) from the plurality of operating modes for which item of identification information. The specified determination and/or selection rules are provided, f r example, in the form of one or more tables, which, for example, assign an item of operating infor-mation to an item of identification information and/or a com-bination of items of identification information. For example, one or more representations of such tables are stored in the server device and/or in a token. It is also conceivable that the token, for example by means of an app, can access determi-nation and/or selection rules stored in the server device.

The method preferably comprises operating, at least partially in dependence on the stored operating information, the elec-trosuraical generator. The operation of the electrosurgical generator then takes place in dependence on the operating mode for which the determined, received, and stored operating information is indicative.

For the case in which no operating mode is available to an electrosurgical generator, an item of operating information can be determined which comprises an item of information in-terpretable accordingly by the electrosurgical generator, and a corresponding message can be displayed to the user, for ex-ample, by means of a suitable interface (for example by dis-play on a display screen).

In addition, the operation of the electrosurgical generator can comprise the control of the energy supply of an electro-surgical instrument.

The energy supply of an electrosurgical instrument is effectu-ated by the electrosurgical generator. The control of the en-eray supply then accordingly takes place at least partially in dependence on the determined, received, and stored operating mode. It is conceivable that a first operating mode specifies a time-dependent power delivery of the energy supply, and a second operating mode regulates a voltage delivery adapted thereto. The first and the second operating modes can then at least partially overlap in time.

The sending of the identification information preferably fur-thermore comprises the steps:
sending, to a token, the identification information;
receiving, at the token, the identification infor-mation;
determining, by way of the token and at least partially in dependence on the identification information, an item of operating information indicative of one operating mode of a plurality of predefined operating modes, wherein the operating modes are each suitable for at least partially controlling an energy supply of an electrosurgical instrument by way of an electrosurgical generator; and
sending, from the token to a server device, the config-uration request comprising the identification infor-mation.

A token is to be understood as a device which is configured in software and/or hardware for the purpose of carrying out the abovementioned method steps. A corresponding suitable generic user terminal can be provided as the token, however, a device designed exclusively for the operation of an electrosurgical generator, for example a correspondingly designed ultrasmall computer (for example a system-on-a-chip) or a chip card can also be provided. The user terminal is, for example, a comput-er such as a desktop computer, a laptop computer, or a tablet computer, or a mobile communication device such. as a smart phone. Corresponding program software or an app, for example, can be installed on the token to carry out the method. The program or the app can have, for example, access to represen-tations of the plurality of operating modes which are stored in a suitable database of the token.

It is obvious that the token can also be configured for pro-cessing an item of request information as described above.

In one embodiment, the method comprises sending, from a token, a preliminary request comprising an item of preliminary infor-mation, and receiving, at the electrosurgical generator, the preliminary request, wherein the sending of the configuxation request takes place in dependence on the preliminary infor-mation. These optional steps should accordingly precede the sending of the configuration request in this way, the method for operating the electrosurgical generator can also be trig-gered by a token.

For example, the preliminary information is characteristic for an identity of the token. This makes it possible that the electrosurgical generator sends the identification information in the context of sending the configuration request to a token which is known to it and trustworthy, and to which, for exam-ple, a secure communication connection already exists. It is also conceivable that the preliminary request initiates the establishment of a secure communication connection between token and electrosurgical generator. The preliminary infor-mation, which is sent by the token and received at the generator in the context of the preliminary request, can then com-prise a coupling request from the token to the generator. In a further step, the generator then sends a coupling confirmation to the token. A challenge-response method can be carried out here, for example, to establish a secure communication connec-tion.

The method can advantageously comprise providing an item of report information, which is indicative of one or more items of operating information stored in the data, memory of the electrosurgical generator.

On the basis of such an item of report information, it can be documented which items of operating information are stored on an electrosurgical generator and provided if necessary for operation. The provided report information can be, for example, presented in a further step directly at the generator to a us-er by means of a suitable report interface or sent to the server device and/or to a token. It is also conceivable that the report information is used in the context of a model for payment for the storage of an item of operating information. Thus, for example, the producer can, on the basis of an item of report information provided to him, for example, by trans-fer to a producer-side server device, track the provision and transfer of one or more items of operating information and in-voice if necessary.

In one embodiment, the determination of the operating infor-mation additionally takes place in dependence on an item of request information, which is indicative of a specific operating mode.

It can be provided, for example, for the case that in depend-ence on an item of identification information, multiple operating modes are suitable for controlling the energy supply or the electrosurgical instrument by the electrosurgical generator, that the determination is also dependent on one or more further items of information present on the server device, for example, received items of information. For example, an item of information about a user input that took place in the electrosurgical generator or in a token, for example a selection of a specific operating mode from a plurality of operating modes by means of the communication interface can be received at the server device in the form of an item of request infor-mation.

Such an item of request information represents here a request for a specific operating mode, independently of whether or not it is permissible for an electrosurgical generator. For exam-ple, the electrosurgical generator or a token presents all operating modes, using which the identified electrosurgical gen-erator can be operated, by means of a suitable interface (for example by display on a display screen) to a user, for example in the context of a user dialogue. The electrosurgical generator or the token can then register a user input, which com-prises a specification of a value and/or a selection from one or more selection options (for example a multiple-choice que-ry). The determination of the operating information can thus provide a first step in which, from the plurality of the provided operating modes, all operating modes suitable fur controlling the energy supply of an electrosurgical instrument by way of the electrosurgical generator are selected and present-ed to the user in a suitable manner. Thereupon, the reception of an item of request information indicative of a user input carried out at a suitable interface having the selection of a specific operating mode from the suitable operating modes takes place at the server device or also at the token. In a second step of determining the operating information, the re-quest information is also taken into consideration, and an item of operating information is determined which is indicative of the operating mode selected by the user input.

In addition, the request information can be comprised by the configuration request.

Accordingly, the configuration request can already also comprise the request information in addition to the identification information. The electrosurgical generator or a token accepts an item of request information, which is indicative, for example, of a correspondingly registered user input, in the configuration request to be sent. The determination of the operating information then takes place, for example, in a first step in consideration of the request information and only in a second step in consideration of the identification information. If the requested specific operating mode is not available for an electrosurgical generator, but another operating mode is, the operating information can comprise an item of information interpretable accordingly by the electrosurgical generator. For example, the user can cause the renewed sending of a communication request, now with a changed item of request information, for example again on the basis of a user dialogue.

In this way, a user can directly select a specific operating mode for operating an electrosurgical generator.

In a further embodiment, the reception, the determination, and/or the sending of the operating information comprises a check, at least partially in dependence on the identification information, of the permissibility of the operating mode for the electrosurgical generator, wherein the operating information is stored upon positive determination, in particular only upon positive determination, of the permissibility.

To check the permissibility, the electrosurgical generator, the server device, and/or a token can have correspondingly stored items of information which are, for example, indicative of the permissibility of combinations of items of identification information and operating modes.

This procedure corresponds to a check as to whether an operating mode is also permitted for a specific generator, which is identifiable on the basis of the identification information. This check can be carried out by the electrosurgical generator and/or by a further entity at the location of the generator, without access to the server device being required. Similarly, the server device can alternatively or additionally perform such a check.

In the case of a permissibility check by the token, it is conceivable that first and preferably only upon a positive check result is a configuration request sent at all to the server device. It can thus already be checked without the complex configuration of a sufficiently secure and stable communication connection of the generator to the server device whether an individual operating mode is permitted at all for a specific generator.

It is to be established by checking the permissibility whether an operating mode that is fundamentally suitable for the operation of the electrosurgical generator is also permitted for use with the electrosurgical generator, for example by the producer and/or with regard to local conditions such as an existing or absent permissibility of an operating mode for that location at which the generator is to be used. For example, the operating mode can characterize a voltage of the energy supply to be provided by the electrosurgical generator, which the generator can make available based on its design, but is unsuitable for the energy supply of an electrosurgical instrument, for example, due to a deficiency of the instrument only established after the installation of the generator. Further-more, it is conceivable that an operating mode that is implementable in hardware is not permitted in a country, however; this enables a country-specific differentiation. By way of such an additional check on the basis of the identification information of the generator, in spite of the server-side pro-vision of all operating modes available for an identified electrosurgical generator, the individual permissibility of an operating mode can be checked.

If a negative permissibility is established upon the check, a representative item of information interpretable accordingly by the electrosurgical generator can be sent to the generator and/or a corresponding message can be displayed to the user via a suitable interface (for example by display on a display screen) of the token or the generator. Because in this case storage of the operating information in the data memory of the electrosurgical generator does not take place, an additional safety level can be implemented to avoid operation of the generator in a non-permissible operating mode. Before any operation of the electrosurgical generator, the permissibility of an operating mode can be checked on the generator side, on the server side, or by a further entity and possibly damaging operation can be avoided.

Alternatively or additionally, the determination and/or the sending of the operating information can comprise a check, at least partially in dependence on the identification information, of the permissibility of the operating mode for the electrosurgical generator, wherein the operating information is sent in the event of positive determination, in particular only in the event of positive determination, of the permissibility.

This permits a check of the permissibility already before the sending of the corresponding operating information, by which a further safety level can be implemented to avoid possibly dam-aging operation of the electrosurgical generator.

The operating mode preferably specifies one or more of the following operating parameters of the electrosurgical generator: a profile form of the energy supply of an electrosurgical instrument to be provided by the electrosurgical generator, a power, voltage, current and/or frequency of the energy supply of an electrosurgical instrument to be provided by the electrosurgical generator, a specific sequence of profile forms, powers, voltages, currents, and/or frequencies of the energy supply of an electrosurgical instrument to be provided by the electrosurgical generator, a lower limit and/or upper limit to be provided by the electrosurgical generator for the energy supply of the electrosurgical instrument, a user-specific set-ting of the electrosurgical generator with respect to the energy supply of the electrosurgical instrument, and/or a soft-ware update of the electrosurgical generator.

The term “profile form.” refers to chronological properties of voltage, current, power and/or frequency. The profile form can be applied periodically. Accordingly, a characteristic time-dependent provision of voltage, current, power and/or frequency can be characterized by a profile form. A profile form can in turn comprise multiple phases on its part, in which differ-ent provisions of voltage, current, power and/or frequency take place. A profile form can thus comprise, for example, a first phase, which characterizes a specific provision of a first voltage and a first current, while a second phase of the profile form characterizes a specific provision of a second voltage changed from the first voltage and a second current changed from the first current. A profile form is also referred to as a mode. Profile forms are typically characterized with respect to their tissue effect. There are thus profile forms which cause a deep coagulation by direct tissue contact or cause a surface coagulation or a cutting effect without tissue contact.

It is conceivable, for example, that in a first profile form. (“SoftCoag mode”) for deep coagulation, a voltage having a continuous waveform having a frequency of 400 kHz, a duty cycle of 100%, and a maximum voltage amplitude of 200 V is specified. Such a profile form results in tissue temperatures between 60° C. and 95° C. without spark formation and is used to dry out tissue with direct tissue contact. The denaturing of proteins accompanying this and the shrinkage of the tissue caused by the drying ultimately result in a coagulation with greater depth effect.

In a second profile form (“SprayCoag mode”) for coagulation, a modulated waveform having a frequency of 20 kHz, a duty cycle of 2500 ns immediately at the beginning of a period, and thus a duty cycle of 5%, and a maximum voltage amplitude of at least 4000 V can be specified. This second profile form re-sults with spark formation in tissue temperatures of greater than 400° C. and is used to dry out tissue without tissue con-tact. The carbonization of the tissue linked thereto results in a large-area, superficial coagulation. For example, the first and the second profile forms are applied for the energy supply of an electrosurgical instrument designed as a ball electrode.

A third profile form for cutting tissue (“PureCut mode”) spec-ifies a continuous waveform having a frequency of: 400 kHz, a duty cycle of 100%, and a maximum voltage amplitude of 600 V. This profile form results with spark format Hon in tissue tem-peratures of greater than 100° C. The vaporization of liquid linked thereto in the treated tissue results in mechanical tearing of the tissue, by which the tissue is severed. This third profile form can be applied, for example, for the energy supply of an electrosurgical instrument designed as a blade electrode.

Transition profile forms can also be specifiable, the duty cy-cle and maximum voltage amplitude of which are each between those of the third and second profile form.

In a further profile form, a cutting effect can be performed with the aid of a plasma. To carry out this profile form, a conductive liquid is applied, which is heated in a first step by high-frequency currents of up to 12 A, so that a gas layer forms. In a second step, the gas layer is ionized due to the high electrical field strength. A plasma results, which is maintained in the further course of the profile form by a cor-responding control of the energy supply of the electrosurgical instrument.

The power, voltage, current and/or frequency to be provided by the electrosurgical generator can be characterized, for exam-ple, in the form of the average or the maximum amplitude of power, voltage, current and/or frequency of the energy supply. Similarly, lower and/or upper limit, for example, a maximum voltage amplitude or power amplitude, can be characterized by a corresponding operating mode of the energy supply. Value ranges settable on the electrosurgical generator of power, voltage, current and/or frequency can also be represented. One operating mode can thus be characteristic, for example, for a power range to be provided by the electrosurgical generator, for example, of 1 W to 120 W, up to 200 W or up to 300 W, wherein an individual power value from the power range is settable on the generator.

Furthermore, an operating mode can specify a specific sequence of profile forms, powers, voltages, currents and/or frequencies of the energy supply. For example, for a specific tissue treatment, for example, in the context of a typical surgical application, an individual and particularly advantageous time-dependent setting and change of profile forms, powers, voltages, currents and/or frequencies can have emerged, which is provided as a corresponding operating mode at the server device.

In addition, it is conceivable that a user stores a user-specific setting, for example, an individual sequence of profile forms, powers, voltages, currents and/or frequencies of the energy supply beforehand on the server device and has up-loaded it, for example, into the database of the server device. The user-specific settings thus stored can then each be provided individually or in various combinations as an operating mode at the server device.

It is also conceivable that an operating mode comprises a software update or firmware update for the electrosurgical generator. For example, an average or maximum amplitude of power, voltage, current or frequency settable by the generator can be changed by such an update.

The operating mode can also specify operating parameters, by means of which the electrosurgical generator can cause an activation of further devices. It can thus be provided, for ex-ample, that during an energy supply of an electrosurgical instrument by the generator, an additional functionality is to be provided in parallel by a further device. For safety rea-sons, the operation of a fume extraction can thus be provided or also prescribed during the energy supply of an electrosurgical instrument.

The operating information received from the electrosurgical generator has advantageously been determined in dependence on an item of identification information, which is indicative of the identity of the electrosurgical generator. In this way, independently of the source of the operating information, a unique assignment of the operating information to a specific generator can be ensured.

The invention also comprises a computer program having program instructions which cause a processor to carry out and/or con-trol the method according to the invention when the computer program is executed on the processor.

The computer program accord ng to the invention is stored, for example, on a computer-readable data carrier.

The invention also relates to an electrosurgical system comprising an electrosurgical generator according to the invention and a server device according to the invention.

The system preferably furthermore comprises a user terminal suitable as a token. Such a user terminal is then configured in software and or hardware for use as a token.

Furthermore, a method is disclosed which comprises the steps of both methods according to the invention, preferably with incorporation of the token.

The above-described embodiments and designs are solely to be understood as examples and are not to restrict the present invention in any way.

The invention is explained in more detail by way of example hereinafter with reference to the appended drawings on the basis of advantageous embodiments. In the figures:

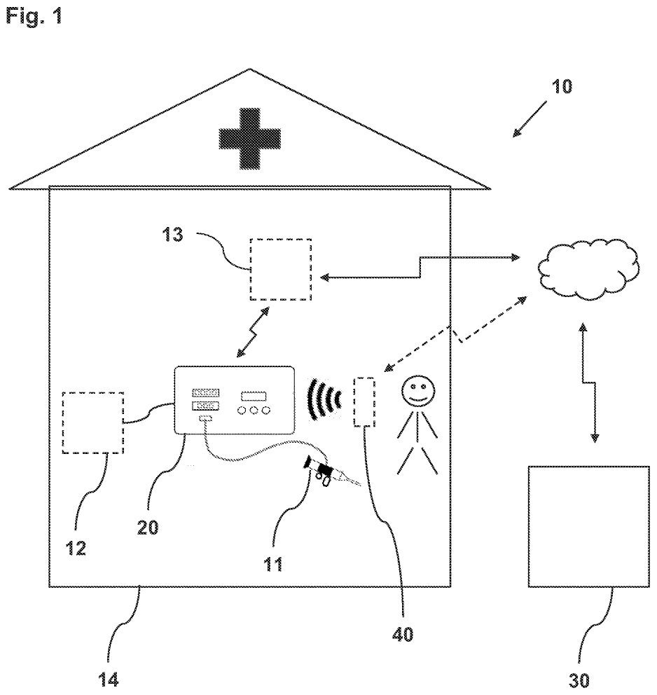

FIG. 1 shows a schematic illustration of an electrosurgical system according to an exemplary embodiment of the invention; and

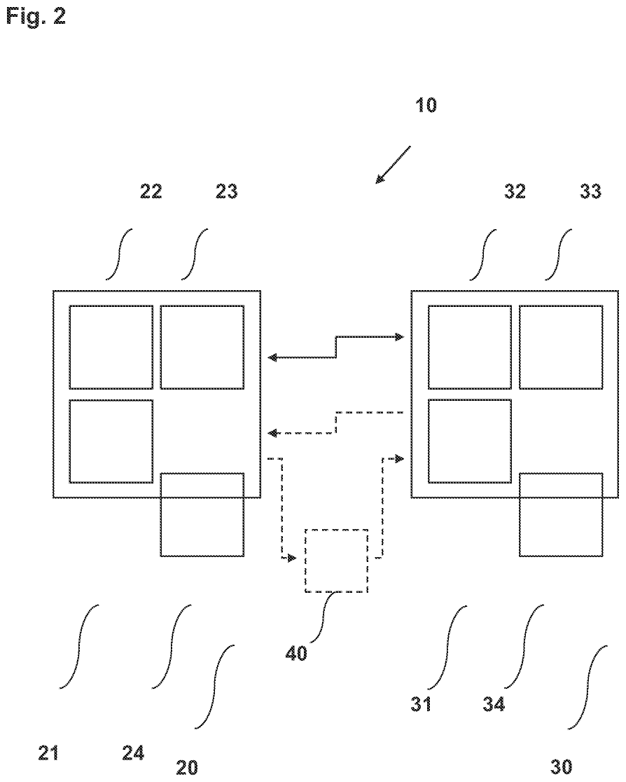

FIG. 2 shows a further schematic illustration of an electrosurgical system, an electrosurgical generator, and a server device according to exemplary embodiments of the invention;

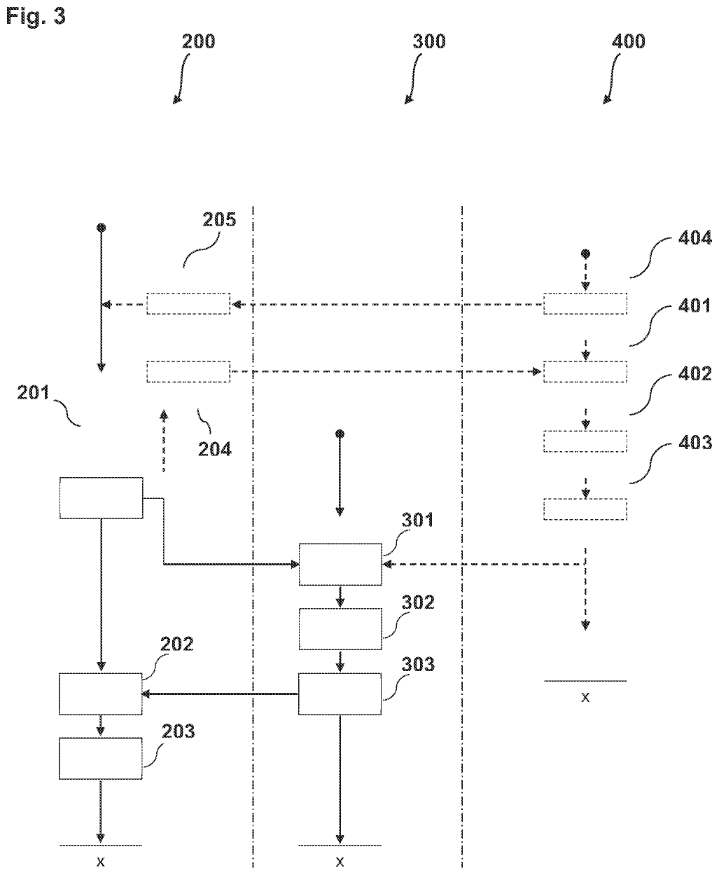

FIG. 3 shows a schematic flow chart of exemplary embodiments of methods according to the invention;

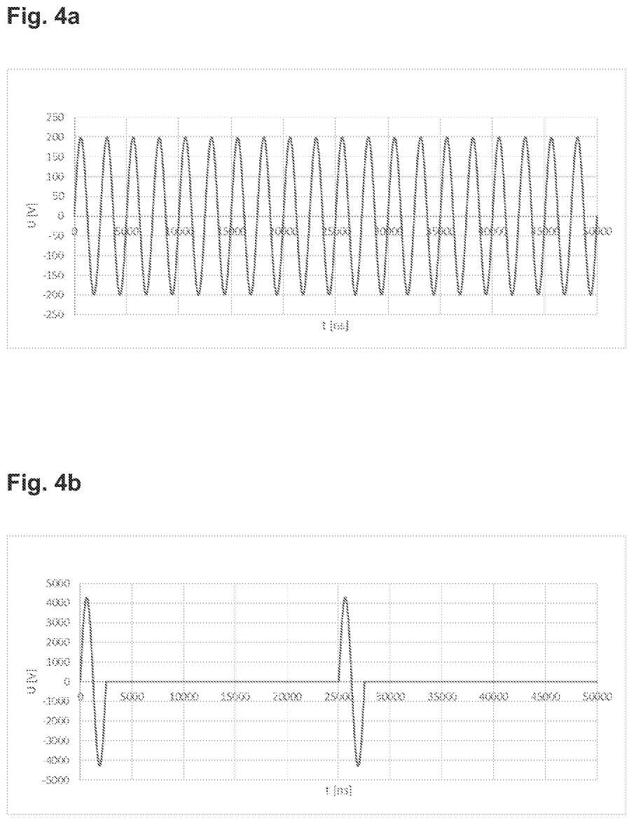

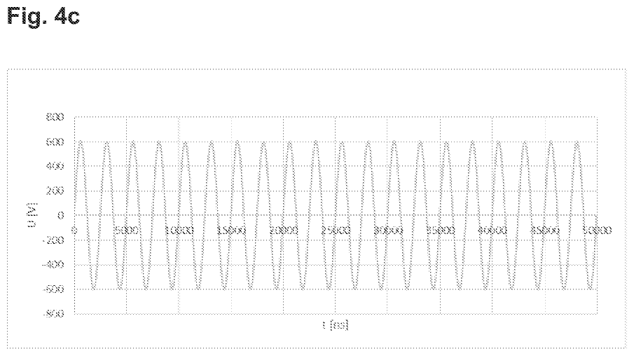

FIG. 4a shows a diagram having a voltage-time curve of an exemplary first t profile form;

FIG. 4h shows a diagram having a voltage-time curve of an exemplary second profile form; and

FIG. 4c shows a diagram having a voltage-time curve of an exemplary third profile form.

FIG. 1 shows a schematic illustration of an electrosurgical system 10. An electrosurgical generator 20 is provided here for the energy supply of an electrosurgical instrument 11 in a hospital 14. The instrument 11 is, for example, an electroscalpel. Furthermore, the system 10 comprises a server device 30 located outside the hospital, in particular at the producer of the electrosurgical generator 20. The electrosurgical generator 20 and the server device 30 can communicate via a communication connection indicated as a cloud, in the present case, for example, the Internet.

Optionally, the communication connection can comprise a node 13 between generator 20 and server device 30 inside the hospital 14, for example in the form of a router. It is conceivable that all communication of components of the hospital 14 to the outside takes place via such a node 13.

The system 10 comprises a user terminal, which is suitable as a token and configured accordingly, in the form of a smart phone 40. The smart phone 40 can communicate for this purpose with the electrosurgical generator 20 via an NFC radio channel and/or both with the electrosurgical generator 20 and also with the server device 30 via the Internet.

A report interface 12 can also optionally be provided in the scope of the system 10. In the present case, this is connected to the electrosurgical generator 20 and can present an item of report information provided there. For this purpose, the interface 12 is designed as a display unit, on which the report information is graphically displayed to a user. It is also conceivable that the report interface 12 alternatively or additionally sends an item of report information via the communication connection, for example, to the server device 30 or the smart phone 40.

FIG. 2 shows a further schematic illustration of an electro-surgical system 10 comprising an electrosurgical generator 20 and a spatially separately arranged server device 30 according to exemplary embodiments.

In addition to typical components (not shown) such as a power supply unit, an inverter, and a connection for an electrosurgical instrument 11, the electrosurgical generator 20 comprises a processor 21, a communication interface 22, a data memory 24, and a data carrier 23. In a similar manner, the server device 30 comprises a processor 31, a communication interface 32, a database 34, and a data carrier 33. The database 34 is designed to store a plurality of items of operating information. The data memory 24 is in turn designed to store one or more items of operating information, which is to be a subset of the plurality of items of operating information storable in the data, memory 34.

The electrosurgical generator 20 and the server device 30 are designed to communicate via a communication connection by means of their communication interfaces 22, 23. In the context of such a communication, a configuration request can be sent from the electrosurgical generator 20 and received by the server device 30, thus transferred. An item of operating information can also be sent by the server device 30 and received by the electrosurgical generator 20 and thus transferred.

The electrosurgical system 10 can preferably comprise a token 40, which is indicated by dashed lines in FIG. 2 together with the corresponding communication connections. The electro-surgical generator 20 can communicate via its communication interface 22, for example via an NFC radio channel, with the token 40 and transfer an item of identification information to the token 40. The token 40 is in turn configured to send a configuration request to the server device 30, which can be received by the communication interface 32. The server device 30 is also configured to transfer an item of operating information to the electrosurgical generator 20 upon use of the token 40.

The designs of the functional modules (processor, communication interface, data memory, database, data carrier) of: the electrosurgical generator 20 and the server device 30 are not described in more detail hereinafter, since they are in the scope of routine measures in the art. It is thus readily possible for a person skilled in the art, for example, to make use of typically used types of memory (RAM, ROM, EEPROM) in the design of the data memory 24, the database 34, and the da-ta carrier 23, 33.

FIG. 3 illustrates exemplary embodiments of methods according to the invention. The method 200 represents an exemplary method for operating an electrosurgical generator and is carried out in the electrosurgical generator 20. Correspondingly, the method 300 represents an exemplary method for operating a server device 30 and is carried out in the server device 30. As is apparent in FIG. 3, both methods are technically linked to one another in the context of transferring a configuration request from the electrosurgical generator 20 to the server device 30 and transferring an item of operating information in the reverse direction. Individual or all steps of the method 400 can optionally be provided, which is carried out in a token 40.

In the method 200, the electrosurgical generator 20 sends a configuration request to a server device 30 in a step 201. The configuration request comprises an item of identification information indicative of the identity of the electrosurgical generator 20 and also the identity of the electrosurgical instrument 11.

The sending 404 and the receiving 205 of a preliminary request preferably precede step 201. The preliminary request, which comprises a preliminary item of information, is sent in step 404 by the token 40 and received in step 205 at the electro-surgical generator 20. The sending of the configuration re-quest in step 201 then takes place in dependence on the received preliminary information. In the present example, it can be established on the basis of the preliminary information for the generator 20 that the token 40 is a trustworthy token, to which a secure communication connection can be established in a manner routine in the art. Then, for example, optional step 204 described hereinafter or also a transfer of an item of re-port information from the generator 20 to the token 40 can take place via this communication connection.

Optionally, step 201 can comprise sending 204 the identification information to the token 40, whereupon steps 401 to 403 of the method 400 are carried out in the token 40. The token 40 receives the identification information in step 401. In step 402, the token 40 determines an item of operating information which is indicative of an operating mode. This can take place similarly to step 302 of the method 300 explained below.

In step 403, the token 40 sends a corresponding configuration request to the server device 30 instead of the electrosurgical generator 20. It is conceivable here that the token already carries out a check of the permissibility of the operating mode for the electrosurgical generator 20 on the basis of the identification information received in step 401 and a configuration request is only sent to the server device according to step 403 at all in the event of a positive check result.

In step 301, the configuration request sent by the electrosurgical generator 20 or by the token 40 is received at the server device 30. This takes place by means of the communication interface 32.

For the server device 30, a specific operating mode for the combination of generator 20 and instrument 11 characterized by the identification information is now determinable on the basis of predefined rules. This is dependent, for example, on whether the generator 20 can set the operating mode in hardware and the in is designed in hardware for the operating mode. For this purpose, the server device 30 has access to a plurality of predefined operating modes stored on a data carrier 33 of the server device 30 and to selection rules, which specify which operating mode from the plurality of operating modes is to be determined for the combination of generator 20 and instrument. 11 represented by the identification information.

In dependence on the identification information, the server device 30 accordingly determines in step 302 an item of operating information, which is indicative of one operating mode of a plurality of predefined operating modes. The operating modes are each capable of at least partially controlling an energy supply of an electrosurgical instrument 11 by the electrosurgical generator 20. For this purpose, the plurality of operating modes each specify one or more operating parameters of the electrosurgical generator 20. Thus, for example, an operating mode can be determined which specifies the third profile form of the energy supply of FIG. 4c for cutting tissue to be provided by the electrosurgical generator 20. In this example, the determined operating mode is also to specify a power range of 1 W to 300 W to be provided by the electrosurgical generator, from which an individual power value is settable at the generator.

The operating information determined in step 302 is sent in step 303 from the server device 30 by means of the communication interface 32 and received in step 202 by the electrosurgical generator 20 at its communication interface 22.

The determination in step 302 can additionally be dependent Cr an item of request information, which the electrosurgical generator 20 registers and transfers to the server device 30. The request information is to be representative of a request of the user for a specific operating mode, which is registered by a corresponding user input at a suitable interface of the electrosurgical generator. For this purpose, the electrosurgical generator 20 enables the user, again by means of a suitable interface, for example a touch-sensitive display unit, to make a selection by user input from a plurality of operating modes, using which the identified electrosurgical generator 20 can be operated. In a second partial step within the determination of step 302, the request information can also be taken into consideration and an item of operating information can be determined, which is also indicative of the specific operating mode requested by the user input.

In step 203, the operating information received in step 202 is stored in a data memory 24 of the electrosurgical generator 20. The generator 20 is preferably operated in a further step at least partially in dependence on the operating information stored in step 202. This comprises, for example, the control of the energy supply of an electrosurgical instrument 11.

FIGS. 4a, 4b and 4c show exemplary profile forms of the energy supply of an electrosurgical instrument 11, which each show a voltage U in V to be provided by the electrosurgical generator 20 in relation to the time t in ns in a diagram.

The profile form in FIG. 4a corresponds to a profile form for deep coagulation. A voltage having a continuous waveform having a frequency of 400 kHz, a duty cycle of 100%, and a maximum voltage amplitude of 200 V is specified. In FIG. 4b, a profile form for coagulation is shown, which specifies a modulated waveform having a frequency of 40 kHz, a duty cycle of 2500 ns immediately at the beginning of a period, and thus a duty cycle of 10%, and a maximum voltage amplitude of greater than 4000 V. The profile form shown in FIG. 4c is a profile form for cutting tissue. This profile form seed ties a continuous waveform having a frequency of 400 kHz, a duty cycle of 100%, and a maximum voltage amplitude of 600 V.

The embodiments of the present invention described in this specification and the optional features and properties respectively set forth in this regard are also to be understood as disclosed in all combinations with one another. In particular, the description of a feature comprised by one embodiment—not explicitly stated otherwise—is not to be understood in the present case so that the feature is indispensable or essential for the function of the embodiment.

1. A method for operating an electrosurgical generator for the energy supply of an electrosurgical instrument, the method comprising:
sending, to a spatially separately arranged server device, a configuration request comprising an item of identification information indicative of an identity of the electrosurgical generator;
receiving an item of operating information indicative of an operating mode, wherein the operating mode is capable of at least partially controlling the energy supply of the electrosurgical instrument by the electrosurgical generator; and
storing the operating information in a data memory of the electrosurgical generator.

2. The method according to claim 1, further comprising:
operating the electrosurgical generator in the operating mode for which the stored operating information is indicative.

3. The method according to claim 2, wherein the operation of the electrosurgical generator comprises the control of the energy supply of an electrosurgical instrument.

4. The method according to claim 1, wherein the sending of the identification information further comprises:
sending, to a token, the identification information;
receiving, at the token, the identification information;
determining, by way of the token and at least partially in dependence on the identification information, an item of operating information indicative of one operating mode of a plurality of predefined operating modes, wherein the operating modes are each capable of at least partially controlling an energy supply of an electrosurgical instrument by an electrosurgical generator; and
sending, from the token to the server device, the configuration request comprising the identification information.

5. The method according to claim 1, further comprising:
sending, from a token, a preliminary request comprising an item of preliminary information; and
receiving, at the electrosurgical generator, the preliminary request, wherein the sending of the configuration request takes place in dependence on the preliminary information.

6. The method according to claim 1, further comprising:
providing an item of report information, which is indicative of one or more items of operating information stored in the data memory of the electrosurgical generator.

7. The method for operating a server device, the method comprising:
receiving a configuration request comprising an item of identification information indicative of an identity of an electrosurgical generator;
determining, at least partially in dependence on the identification information, an item of operating information indicative of one operating mode from a plurality of predefined operating modes, wherein the operating modes are each capable of at least partially controlling an energy supply of an electrosurgical instrument by the electrosurgical generator; and
sending, to the electrosurgical generator, the operating information.

8. The method according to claim 7, wherein the determination of the operating information additionally takes place in dependence on an item of request information, which is indicative of a specific operating mode.

9. The method according to claim 8, wherein the request information is comprised by the configuration request.

10. The method according to claim 1, wherein the reception, the determination, and/or the sending of the operating information comprises a check, at least partially in dependence on the identification information, of the permissibility of the operating mode for the electrosurgical generator, and wherein the operating information is stored in the event of a positive check of the permissibility.

11. The method according to claim 1, wherein the determination and/or the sending of the operating information comprises a check, at least partially in dependence on the identification information, of the permissibility of the operating mode for the electrosurgical generator, and wherein the operating information is sent in the event of a positive check of the permissibility.

12. The method according to claim 1, wherein the operating mode specifies one or more of the following operating parameters of the electrosurgical generator:
a profile form of the energy supply of an electrosurgical instrument to be provided by the electrosurgical generator,
a power, voltage, current and/or frequency of the energy supply of an electrosurgical instrument to be provided by the electrosurgical generator,
a specific sequence of profile forms, powers, voltages, currents and/or frequencies of the energy supply of an electrosurgical instrument to be provided by the electrosurgical generator,
an upper and/or lower limit for the energy supply of an electrosurgical instrument to be provided by the electrosurgical generator,
a user-specific setting of the electrosurgical generator with respect to the energy supply of an electrosurgical instrument, and/or
a software update of the electrosurgical generator.

13. The method according to claim 1, wherein the operating information received by the electrosurgical generator has been determined in dependence on an item of identification information, which is indicative of the identity of the electrosurgical generator.

14. Computer program having program instructions to cause a processor to carry out and/or control the method according to claim 1 when the computer program is executed on the processor.

15. The computer program according to claim 14, which is stored on a computer-readable data carrier.

16. Electrosurgical generator for the energy supply of an electrosurgical instrument having a processor, a communication interface, a data memory for storing one or more items of operating information, and a computer-readable data carrier, wherein instructions are stored in the data carrier which, upon execution by the processor, cause the method according to claim 1 to be carried out.

17. Server device having a processor, a communication interface, a database for storing a plurality of items of operating information, and a computer-readable data carrier, wherein instructions are stored in the data carrier, which, upon execution by the processor, cause the method according to claim 7 to be carried out.

18. Electrosurgical system, comprising an electrosurgical generator according to claim 16 and a server device.

19. The electrosurgical system according to claim 18, further comprising a token designed as a user terminal.
